(19) United States
(12) Patent Application Publication
(10) Pub. No.: US 2026/0176303 A1
(43) Pub. Date: Jun. 25, 2026

(54) BLOCKING PEPTIDES AND RELATED METHODS, FORMULATIONS, AND COMPOSITIONS

(71) Applicant: TONGJI HOSPITAL, TONGJI MEDICAL COLLEGE OF HUAZHONG UNIVERSITY OF SCIENCE AND TECHNOLOGY, Wuhan (CN)

(72) Inventors: Yong FANG, Wuhan (CN); Ding MA, Wuhan (CN); Qinglei GAO, Wuhan (CN); Dengfeng DOU, Chengdu (CN); Xiaoyuan HUANG, Wuhan (CN); Guangnian ZHAO, Wuhan (CN); Xingzhe LIU, Wuhan (CN); Yuanjia WEN, Wuhan (CN)

(73) Assignee: TONGJI HOSPITAL, TONGJI MEDICAL COLLEGE OF HUAZHONG UNIVERSITY OF SCIENCE AND TECHNOLOGY, Wuhan (CN)

(21) Appl. No.: 19/332,100

(22) Filed: Sep. 18, 2025

(30) Foreign Application Priority Data
Dec. 25, 2024 (CN) 202411925138.2

(51) Int. Cl. C07K 7/08; A61K 31/381; A61K 31/4439; A61K 31/502; A61K 38/00; A61P 35/00

(52) U.S. Cl. CPC C07K 7/08; A61K 31/381; A61K 31/4439; A61K 31/502; A61P 35/00; A61K 38/00; C07K 2319/20

(57) ABSTRACT

Disclosed are blocking peptides and related methods, formulations, and compositions, pertaining to the field of biomedicine. Based on the regulatory mechanism by which the negative regulator TGF-β modulates the transport and degradation of STING, the use of AP3D1 as a drug target for in vitro screening of blocking agents is provided to identify the S9 regulatory site and the R26 regulatory site of AP3D1. Corresponding blocking peptides targeting the S9 regulatory site and the R26 regulatory site, respectively, were synthesized to specifically inhibit AP3D1-mediated inactivation of STING signaling by tumor microenvironment factors, thereby blocking at least one of the S9 regulatory site and the R26 regulatory site and effectively restricting STING transport and degradation. The blocking peptide can synergize with various treatment modalities, including chemotherapy, radiotherapy, PARP inhibitors, and STING agonists, significantly enhancing cGAS-STING signaling activation in tumors during therapy and achieving more potent antitumor efficacy.

### CROSS-REFERENCE TO RELATED APPLICATIONS

This application claims priority to Chinese Patent Application No. 202411925138.2, filed on Dec. 25, 2024, the entire contents of which are hereby incorporated by reference.

### SEQUENCE LISTING

The instant application contains a Sequence Listing which is submitted electronically in XML format and is hereby incorporated by reference in its entirety. The XML copy, created on Mar. 19, 2025, is named “2025-3-19-Sequence List-67905-H006US00,” and is 3,811 bytes in size.

### TECHNICAL FIELD

The present disclosure relates to the field of biomedical technology, and particularly to blocking peptides and related methods, formulations, and compositions.

### BACKGROUND

A cyclic GMP-AMP synthase-stimulator of interferon genes (cGAS-STING) innate immune pathway serves as a first line of defense against infections and tumors in humans, wherein a stimulator of interferon genes (STING) is a critical transmembrane adaptor protein responsible for immune responses in the innate immune pathway. During pathogenic infection or tumorigenesis, aberrant deoxyribonucleic acid (DNA) is recognized by the cytosolic nucleic acid receptor cyclic GMP-AMP synthase (cGAS) in host cells. Activated cGAS utilizes adenosine triphosphate (ATP) and guanosine triphosphate (GTP) to synthesize a second messenger molecule cyclic guanosine monophosphate-adenosine monophosphate (cGAMP). The second messenger, cGAMP, acts as a signaling molecule in the innate immune system, binding to and interacting with the endoplasmic reticulum-localized protein STING, thereby inducing conformational changes in STING. Upon activation, STING translocates from the endoplasmic reticulum to the perinuclear endosome along with TANK-binding kinase 1 (TBK1). The TBK1 kinase phosphorylates and activates interferon regulatory factors 3/7 (IRF3/7) and nuclear factor kappa B (NF-κB), leading to the production of type I interferons (IFNs) and other immune response factors. Concurrently, activated STING is transported from the Golgi apparatus to lysosomes for degradation, enabling precise regulation of STING signaling to prevent cell death caused by excessive IFN responses, which may otherwise lead to autoimmune disorders. Current research has confirmed that STING is a key protein in initiating antitumor innate immune responses and holds promise as a next-generation immunotherapy target capable of converting “cold” tumors into “hot” tumors. Consequently, various STING agonists have been developed to directly activate STING, some of which have entered clinical trials as adjuvant therapies for tumor treatment.

However, despite the demonstrated efficacy of these STING agonists in vitro in activating STING and downstream IFN signaling, their clinical translation has proven challenging. To date, the results from these clinical trials have generally been disappointing.

Therefore, it is desirable to provide a blocking peptide and related methods, formulations, and compositions capable of significantly enhancing the activation of intratumoral cGAS-STING signaling during treatment, thereby achieving more effective antitumor efficacy.

### SUMMARY

One or more embodiments of the present disclosure provide a blocking peptide capable of blocking at least one of an S9 regulatory site and an R26 regulatory site of Adaptor-Related Protein Complex 3 Subunit Delta 1 (AP3D1).

In some embodiments, the blocking peptide includes: (a) an amino acid sequence having 90% to 99% sequence identity to at least one of: (1) the amino acid sequence set forth in SEQ ID NO: 1, and (2) the amino acid sequence set forth in SEQ ID NO: 2; or (b) a combination of amino acid sequences, wherein each sequence in the combination independently satisfies (a).

In some embodiments, the blocking peptide includes: (a) an amino acid sequence selected from at least one of: (1) the amino acid sequence set forth in SEQ ID NO: 1, and (2) the amino acid sequence set forth in SEQ ID NO: 2; or (b) a combination of amino acid sequences, wherein each sequence in the combination independently satisfies (a).

In some embodiments, the blocking peptide includes: (a) an amino acid sequence selected from at least one of: (1) the amino acid sequence set forth in SEQ ID NO: 1, and (2) the amino acid sequence set forth in SEQ ID NO: 2, wherein the selected amino acid sequence has one or more modifications selected from: (i) substitution of one or more amino acids, (ii) deletion of one or more amino acids, and (iii) addition of one or more amino acids; or (b) a combination of amino acid sequences, wherein each sequence in the combination independently satisfies (a).

In some embodiments, the blocking peptide includes: (a) an amino acid sequence selected from at least one of: (1) the amino acid sequence set forth in SEQ ID NO: 1, (2) the amino acid sequence set forth in SEQ ID NO: 2, and (3) the amino acid sequence set forth in SEQ ID NO: 3, which is included within at least one of SEQ ID NO: 1 and SEQ ID NO: 2, wherein the selected amino acid sequence is conjugated with: a tag or a modifying group at an N-terminus, a C-terminus, or both the N-terminus and the C-terminus; or (b) a combination of amino acid sequences each independently selected from (a)(1)-(3) and conjugated as defined in (a).

In some embodiments, the blocking peptide includes: (a) an amino acid sequence selected from at least one of: (1) the amino acid sequence set forth in SEQ ID NO: 1, and (2) the amino acid sequence set forth in SEQ ID NO: 2, wherein the selected amino acid sequence has one or more modifications selected from: (i) substitution of one or more amino acids, (ii) deletion of one or more amino acids, and (iii) addition of one or more amino acids, and the modified sequence is conjugated with: a tag or a modifying group at an N-terminus, a C-terminus, or both the N-terminus and the C-terminus; or (b) a combination of amino acid sequences each independently satisfying the conditions of (a).

One or more embodiments of the present disclosure provide a peptide formulation. The peptide formulation includes the aforementioned blocking peptide.

In some embodiments, the peptide formulation further includes a pharmaceutically acceptable excipient.

One or more embodiments of the present disclosure provide a tumor-inhibiting composition. The tumor-inhibiting composition includes: (a) the polypeptide formulation above; and (b) a Poly(ADP-ribose) polymerase (PARP) inhibitor or a stimulator of interferon genes (STING) agonist.

In some embodiments, the PARP inhibitor includes niraparib or olaparib.

In some embodiments, the STING agonist includes MSA-2.

In some embodiments, the tumor includes an ovarian tumor, a breast tumor, or a colon tumor.

### BRIEF DESCRIPTION OF THE DRAWINGS

The patent or application file contains at least one drawing executed in color.

Copies of this patent or patent application publication with color drawing(s) will be provided by the Office upon request and payment of the necessary fee.

The present disclosure will be further illustrated by way of exemplary embodiments, which will be described in detail with reference to the accompanying drawings. These embodiments are not limiting. In these embodiments, the same numbering indicates the same structure, wherein:

FIG. 1 is a schematic structural diagram of blocking peptides according to some embodiments of the present disclosure;

FIG. 2 shows immunoblot (Western blot) detection results of blocking peptides according to some embodiments of the present disclosure;

FIG. 3 shows in vivo bioluminescence imaging of mice in each experimental group at a 4th week after administration of blocking peptides according to some embodiments of the present disclosure;

FIG. 4 is a comparative graph of tumor burden in mice from each experimental group at a 4th week after administration of blocking peptides according to some embodiments of the present disclosure;

FIG. 5 is a comparative graph of ascites volume in mice from each experimental group at a 4th week after administration of blocking peptides according to some embodiments of the present disclosure;

FIG. 6 is a comparative graph of mammary tumor volume changes in mice from each experimental group during treatment with blocking peptides according to some embodiments of the present disclosure;

FIG. 7 is a comparative photographic representation of mammary tumor sizes in mice from each experimental group during treatment with blocking peptides according to some embodiments of the present disclosure;

FIG. 8 is a comparative graph of mammary tumor weight changes in mice from each experimental group during treatment with blocking peptides according to some embodiments of the present disclosure;

FIG. 9 is a comparative graph of subcutaneous colon tumor volume changes in mice from each experimental group during treatment with blocking peptides according to some embodiments of the present disclosure;

FIG. 10 is a comparative photographic representation of subcutaneous colon tumor sizes in mice from each experimental group during treatment with blocking peptides according to some embodiments of the present disclosure;

FIG. 11 is a comparative graph of subcutaneous colon tumor weight changes in mice from each experimental group during treatment with a blocking peptide according to some embodiments of the present disclosure;

FIG. 12 is a comparative graph of IFN signaling status, illustrating an application of AP3D1 according to some embodiments of the present disclosure;

FIG. 13 is a comparative graph of differential gene expression in OVCAR8 cells under four conditions—control (ctrl), PARP inhibitor Niraparib alone, TGF-β alone, and a combination of Niraparib and TGF-β—demonstrating an application of AP3D1 according to some embodiments of the present disclosure;

FIG. 14 is a comparative graph of differential gene expression under four conditions—control (ctrl), STING agonist MSA-2 alone, TGF-β alone, and a combination of MSA-2 and TGF-β—demonstrating an application of AP3D1 according to some embodiments of the present disclosure;

FIG. 15 is a Western blot (WB) analysis of an interaction between TGF-β and a STING agonist MSA-2, illustrating an application of AP3D1 according to some embodiments of the present disclosure;

FIG. 16 is an immunofluorescence image of an interaction between TGF-β and a STING agonist MSA-2, demonstrating an application of AP3D1 according to some embodiments of the present disclosure;

FIG. 17 is a Venn diagram showing intersections of: (i) all proteins binding to STING after ctrl or TGF-β pretreatment, (ii) phosphorylated proteins post-TGF-β treatment from public databases, and (iii) STING transport-related proteins from the Gene Ontology (GO) database, illustrating an application of AP3D1 according to some embodiments of the present disclosure;

FIG. 18 is a Western blot (WB) analysis of interaction sites between AP3D1 and STING, demonstrating an application of AP3D1 according to some embodiments of the present disclosure; and

FIG. 19 is a schematic workflow diagram for constructing STING-AP3D1 site-directed mutant plasmids and their detection, illustrating an application of AP3D1 according to some embodiments of the present disclosure.

### DETAILED DESCRIPTION

In order to further illustrate the technical solutions of the embodiments of the present disclosure, a brief introduction will be made to the drawings required for the description of the embodiments. It is obvious that the drawings described below are only examples or embodiments of the present disclosure. For those skilled in the art, without exercising inventive labor, the present disclosure may also be applied to other similar scenarios based on these drawings. Unless otherwise indicated or specified from the context, identical reference numerals in the drawings represent identical structures or operations.

One or more embodiments of the present disclosure provide a blocking peptide capable of blocking at least one of an S9 regulatory site and an R26 regulatory site of Adaptor-Related Protein Complex 3 Delta 1 Subunit (AP3D1).

The blocking peptide is a short peptide sequence that specifically binds to AP3D1. In some embodiments, a binding site of the blocking peptide to AP3D1 may be at least one of the S9 regulatory site and the R26 regulatory site, thereby blocking at least one of the S9 regulatory site and the R26 regulatory site of AP3D1.

In some embodiments, the blocking peptide may be used in the preparation of a polypeptide formulation.

In some embodiments, the blocking peptide may include: (a) an amino acid sequence having 90% to 99% sequence identity to at least one of: (1) the amino acid sequence set forth in SEQ ID NO: 1, and (2) the amino acid sequence set forth in SEQ ID NO: 2; or (b) a combination of amino acid sequences, wherein each sequence in the combination independently satisfies (a).

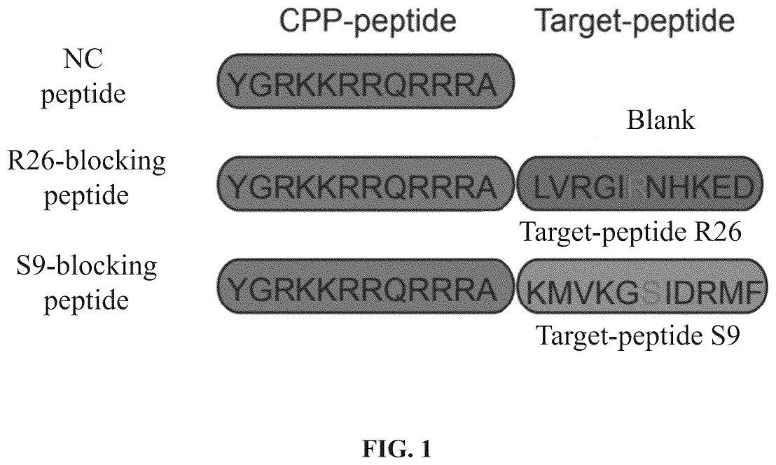

In some embodiments, SEQ ID NO: 1 is an S9-blocking peptide. For example, as shown in FIG. 1, SEQ ID NO: 1 may be:

YGRKKRRQRRRAKMVKGSIDRMF.

Alternatively SEQ ID NO: 1 may be represented as

Tyr Gly Arg Lys Lys Arg Arg Gln Arg Arg Arg Ala
Lys Met Val Lys Gly Ser Ile Asp Arg Met Phe.

In some embodiments, SEQ ID NO: 2 is an R26-blocking peptide. For example, as shown in FIG. 1, SEQ ID NO: 2 may be:

YGRKKRRQRRRALVRGIRNHKED.

Alternatively, SEQ ID NO: 2 may be represented as

Tyr Gly Arg Lys Lys Arg Arg Gln Arg Arg Arg Ala
Leu Val Arg Gly lle Arg Asn His Lys Glu Asp.

It should be noted that in the above two polypeptide sequences, the underlined portion (YGRKKRRQRRRA) corresponds to the cell-penetrating peptide (CPP-peptide) sequence, as labeled in FIG. 1. The remaining segments represent a tag peptide (also referred to as a targeting peptide), which is a short peptide sequence. The CPP sequence (Tyr Gly Arg Lys Lys Arg Arg Gln Arg Arg Arg Ala) may be denoted as SEQ ID NO: 3, which is included in both SEQ ID NO: 1 and SEQ ID NO: 2. For the S9-blocking peptide, the tag peptide is an S9-targeting peptide, wherein the Ser (S) residue corresponds to the S9 regulatory site. For the R26-blocking peptide, the tag peptide is an R26-targeting peptide, wherein the second Arg (R) residue corresponds to the R26 regulatory site. An NC peptide refers to a polypeptide consisting solely of the CPP sequence (SEQ ID NO: 3) without any tag peptide.

The term “having 90% to 99% sequence identity” means that the blocking peptide includes an amino acid sequence that, when aligned with SEQ ID NO: 1 or SEQ ID NO: 2, shares 90% to 99% identical amino acid residues.

In some embodiments, the blocking peptide may include: (a) an amino acid sequence selected from at least one of: (1) the amino acid sequence set forth in SEQ ID NO: 1, and (2) the amino acid sequence set forth in SEQ ID NO: 2; or (b) a combination of amino acid sequences, wherein each sequence in the combination independently satisfies (a).

In some embodiments, the blocking peptide may include: (a) an amino acid sequence selected from at least one of: (1) the amino acid sequence set forth in SEQ ID NO: 1, and (2) the amino acid sequence set forth in SEQ ID NO: 2, wherein the selected amino acid sequence has one or more modifications selected from: (i) substitution of one or more amino acids, (ii) deletion of one or more amino acids, and (iii) addition of one or more amino acids; or (b) a combination of amino acid sequences, wherein each sequence in the combination independently satisfies (a).

The term “substitution of one or more amino acids” refers to a replacement of one or more amino acids in the sequence set forth in SEQ ID NO: 1 or the sequence set forth in SEQ ID NO: 2 with other amino acids.

The term “deletion of one or more amino acids” refers to a removal of one or more amino acids from the sequence set forth in SEQ ID NO: 1 or the sequence set forth in SEQ ID NO: 2.

The term “addition of one or more amino acids” refers to an insertion of one or more extra amino acids into the sequence set forth in SEQ ID NO: 1 or the sequence set forth in SEQ ID NO: 2.

In some embodiments, the blocking peptide may include: (a) an amino acid sequence selected from at least one of: (1) the amino acid sequence set forth in SEQ ID NO: 1, (2) the amino acid sequence set forth in SEQ ID NO: 2, and (3) the amino acid sequence set forth in SEQ ID NO: 3, which is included within at least one of SEQ ID NO: 1 and SEQ ID NO: 2, wherein the selected amino acid sequence is conjugated with: a tag or a modifying group at an N-terminus, a C-terminus, or both the N-terminus and the C-terminus; or (b) a combination of amino acid sequences each independently selected from (a)(1)-(3) and conjugated as defined in (a).

The tag refers to a short peptide, an amino acid sequence, or a molecule that may be fused to the amino acid sequence set forth in SEQ ID NO: 1 or SEQ ID NO: 2 for purposes such as purification, detection, or localization. It should be noted that the tag herein is distinct from the aforementioned tag peptide.

The modifying group refers to a chemical moiety attached to the amino acid sequence set forth in SEQ ID NO: 1 or SEQ ID NO: 2 to alter a property, a function, or a stability thereof.

The phrase “at an N-terminus, a C-terminus, or both the N-terminus and the C-terminus” refers to that the tag or the modifying group is conjugated to at least one of the N-terminal (i.e., the amino terminus) and the C-terminal (i.e., the carboxyl terminus) of the amino acid sequence set forth in SEQ ID NO: 1 or SEQ ID NO: 2.

In some embodiments, the blocking peptide may include: (a) an amino acid sequence selected from at least one of: (1) the amino acid sequence set forth in SEQ ID NO: 1, and (2) the amino acid sequence set forth in SEQ ID NO: 2, wherein the selected amino acid sequence has one or more modifications selected from: (i) substitution of one or more amino acids, (ii) deletion of one or more amino acids, and (iii) addition of one or more amino acids, and the modified sequence is conjugated with: a tag or a modifying group at an N-terminus, a C-terminus, or both the N-terminus and the C-terminus; or (b) a combination of amino acid sequences each independently satisfying the conditions of (a).

In some embodiments, the blocking peptide may further include an additional amino acid sequence. For example, the blocking peptide may include any combination of the aforementioned amino acid sequences.

Adaptor-Related Protein Complex 3 Subunit Delta 1 (AP3D1) is a subunit of the Adaptor Protein Complex 3 (AP-3), involved in intracellular protein sorting and vesicular trafficking processes.

The present disclosure provides an application of AP3D1 as a drug target for in vitro screening of a blocker, wherein the blocker refers to a substance capable of inhibiting or blocking a specific biomolecule or a signaling pathway. The blocker may include the aforementioned blocking peptide.

The present disclosure provides a method for in vitro screening of a blocker, comprising: providing candidate blocking peptides; using at least one of the S9 regulatory site and the R26 regulatory site of AP3D1 as a target; and screening from the candidate blocking peptides to identify a target blocking peptide capable of blocking at least one of the S9 regulatory site and the R26 regulatory site. The candidate blocking peptides refer to blocking peptides to be screened, which may be artificially designed. The target blocking peptide refers to a screened blocking peptide.

Regarding the mechanism by which the two regulatory sites of AP3D1 described in the present disclosure may serve as drug targets, an experimental validation process is as follows:

### (1) Identification of STING Signaling Negative Regulator Transforming Growth Factor-Beta (TGF-β)

Given the observation that STING agonists effectively activate STING in vitro but poorly in vivo, it may be hypothesized that a key factor promoting STING transport and degradation may exist within a tumor microenvironment in vivo, which may lead to a scenario where the STING agonists exhibit promising antitumor efficacy in preclinical studies but fail to effectively activate STING signaling in actual tumors.

Therefore, leveraging a previously conducted global first-in-class Phase II clinical trial of PARP inhibitor neoadjuvant therapy for ovarian cancer (NCT04507841), multi-omics analyses including single-cell RNA sequencing (scRNA-seq) were performed on 67 tissue samples from 34 patients with homologous recombination deficiency (HRD) test results, to explore the impact of tumor microenvironment factors on cGAS-STING-IFN signaling before and after PARP inhibitor treatment.

The results revealed that compared to HRD-negative patients, tumor cells from HRD-positive patients exhibited significant enrichment of IFN signaling and higher activity of the cGAS/STING pathway. However, a subset of HRD-positive ovarian cancer patients showed no activation of IFN signaling.

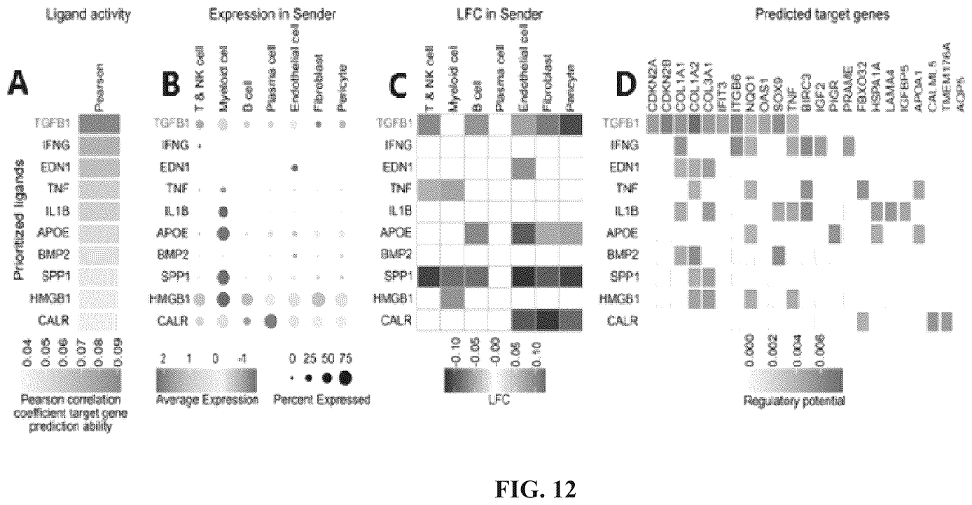

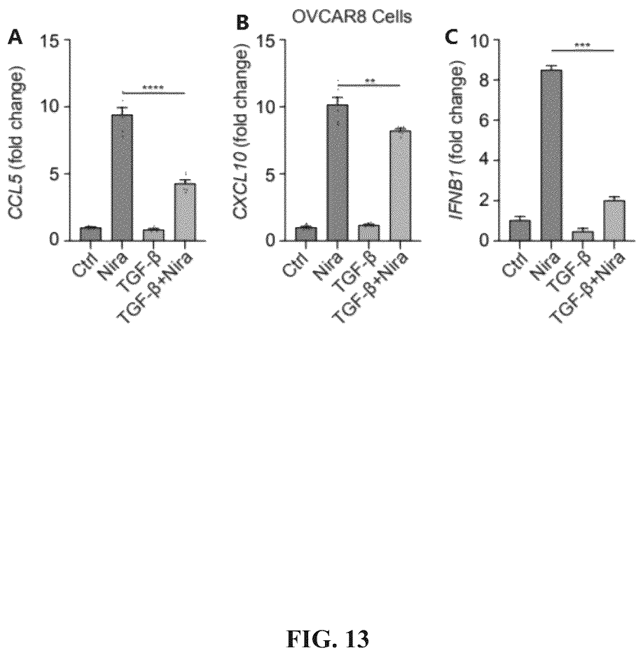

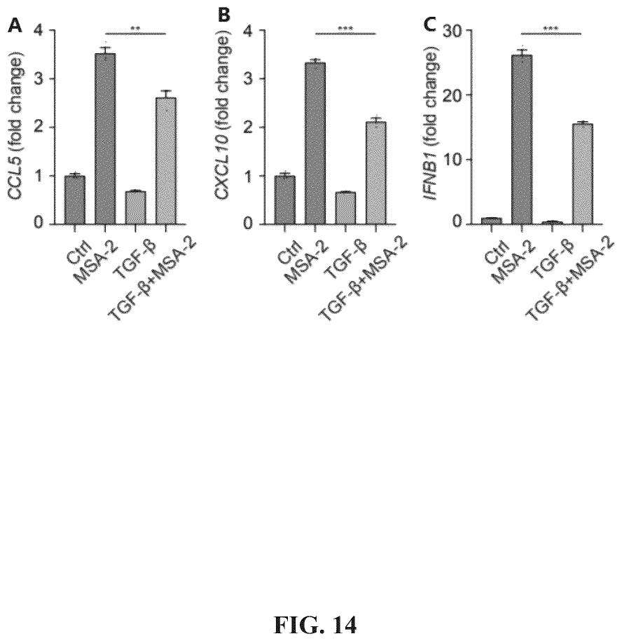

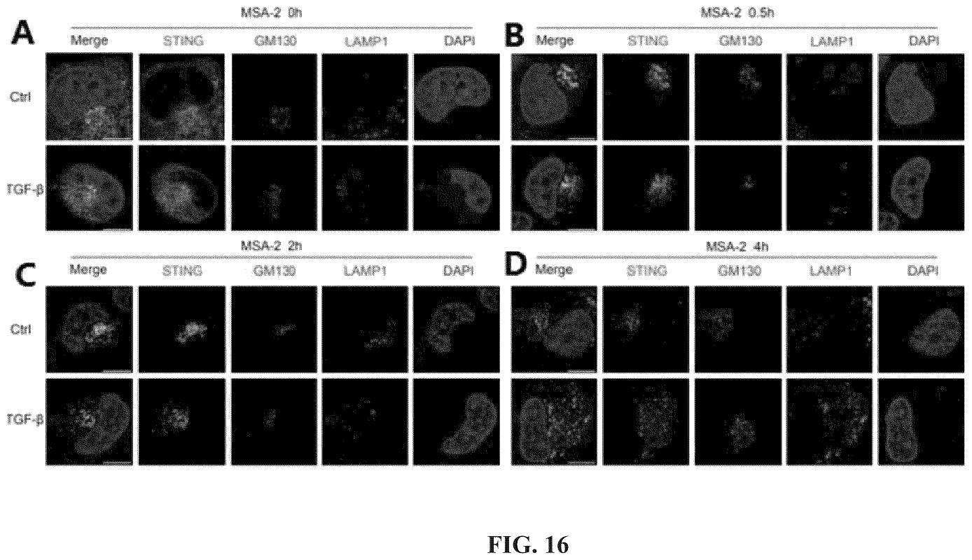

In FIG. 12, Panel A is a potential regulatory ligand activity diagram; Panel B is comparison of expression levels of various regulatory factors across different cell subtypes; Panel C shows differential expression of regulatory factors between an IFN-high cell subtype and an IFN-low cell subtype; and Panel D is a correlation comparison diagram of predicted target genes. In FIG. 13, Panel A represents CCL5; Panel B represents CXCL10; Panel C represents IFNB1. In FIG. 14, Panel A represents CCL5; Panel B represents CXCL10; Panel C represents IFNB1. In FIG. 16, Panel A corresponds to 0 h; Panel B corresponds to 0.5 h; Panel C corresponds to 2 h; and Panel D corresponds to 4 h.

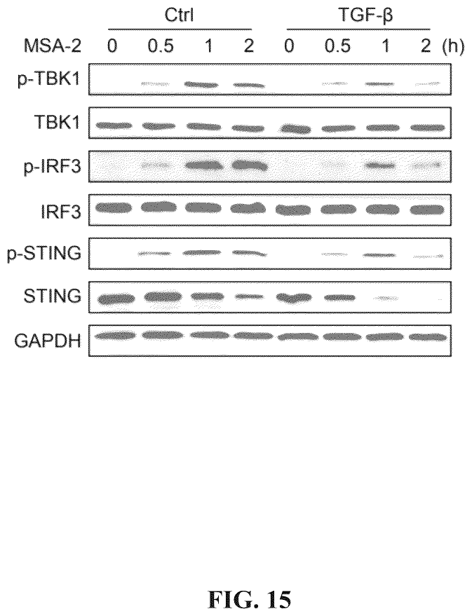

Based on these findings, HRD-positive ovarian cancer patients were divided into two groups (an IFN-high expression group and an IFN-low expression group) according to their IFN signaling status. As shown in FIG. 12, cell communication analysis revealed that tumor cells in the IFN-low expression group received significantly more exogenous TGF-β signals compared to those in the IFN-high group. FIGS. 13 and 14 demonstrate through cytological experiments that TGF-β further suppresses PARP inhibitor-induced and STING agonist-induced STING-IFN signaling post-treatment. As illustrated in FIGS. 15 and 16, Western blot (WB) and immunofluorescence experiments confirmed that TGF-β promotes STING degradation by accelerating its transport from the Golgi apparatus to lysosomes.

These results indicate that TGF-β serves as a key negative regulator of STING signaling in the tumor microenvironment of HRD-positive ovarian cancer, accelerating the transport of activated STING and thereby promoting its degradation.

### (2) Mechanism of TGF-β in Promoting STING Transport and Degradation

To investigate the mechanism by which TGF-β promotes STING transport and degradation, this example constructed a Flag-tagged STING-APEX2 plasmid. The plasmid is capable of identifying all electron-rich, exposed amino acid residues within a 20-nm radius around the STING molecule, including both directly interacting proteins and adjacent proteins. After immunoprecipitation with streptavidin-coated magnetic beads, mass spectrometry is performed to detect differences in STING-interacting molecules with or without TGF-β pretreatment upon STING activation.

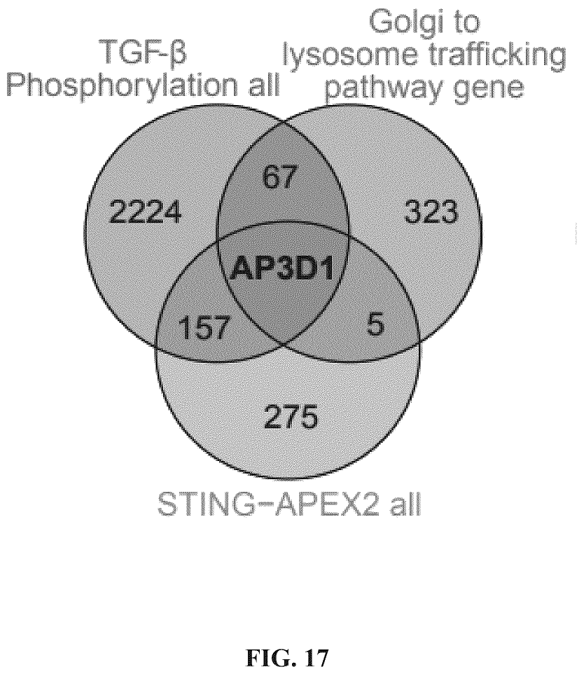

Given that phosphorylation is the most common post-translational modification and TGF-β may function by acting on molecules involved in STING transport and degradation after activation, as shown in FIG. 17, the analysis intersected: (i) all proteins binding to STING after control or TGF-β pretreatment, (ii) phosphorylated proteins following TGF-β treatment from public databases, and (iii) STING transport-related proteins from the Gene Ontology database (GO). The analysis identified AP3D1 as a potential regulatory factor mediating the effects of TGF-β on STING.

### (3) Specific Interaction Sites Between TGF-β and AP3D1, and Between AP3D1 and STING

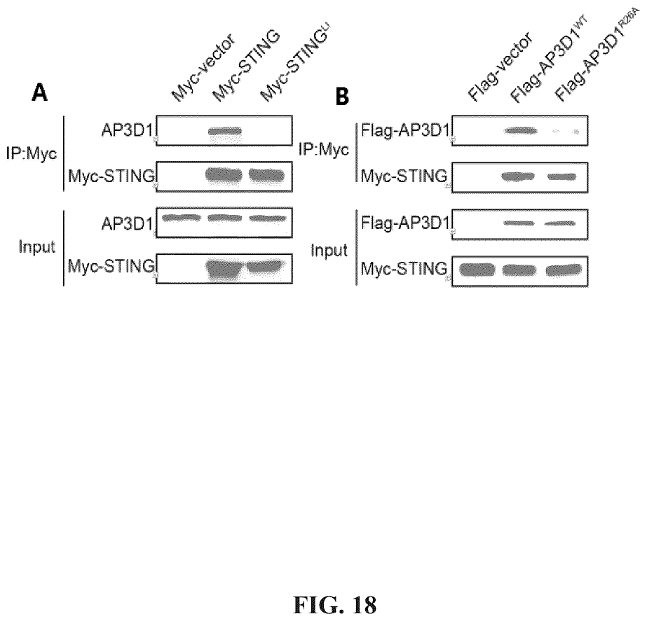

In FIG. 18, Panel A shows the Western blot (WB) analysis of specific binding sites between STING and AP3D1; Panel B displays the WB analysis of interaction sites between the R26 regulatory site of AP3D1 and STING. Existing literature has reported that the AP-1 transport protein recognizes the 360-365 amino acid sequence [D/E]XXXL[L/I] of STING through an R15 site of the AP1γ subunit. The AP-3 transport protein AP3δ subunit shares structural similarity with the AP1γ subunit. Given the homology between AP3D1 and AP1G1, sequence analysis (FIG. 18) revealed that the R26 regulatory site of AP3D1 corresponds to the R15 site of AP1G1 and is highly conserved across multiple species, suggesting the R26 regulatory site as a potential interaction site with the STING [D/E]XXXL[L/I] motif, which is experimentally validated by constructing a STING-AP3D1 site-directed mutant plasmid.

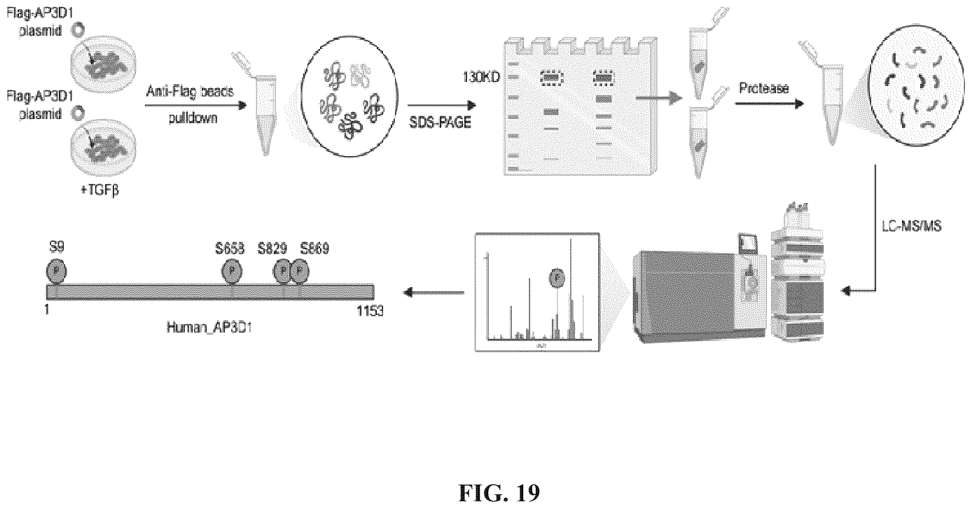

Since components of TGF-β signaling transduction are typically regulated by phosphorylation, to identify a TGF-β3-induced phosphorylation site on AP3D1, phosphoproteomic analysis combined with experimental validation (FIG. 19) reveals that the S9 regulatory site of AP3D1 is the potential TGF-β phosphorylation site under ctrl/TGF-β treatment.

Based on these experimental results, it is confirmed that TGF-β promotes phosphorylation of the S9 regulatory site of AP3D1, thereby enhancing the interaction between the AP3D1 R26 regulatory site and STING, ultimately accelerating STING transport and degradation.

Therefore, to inhibit STING transport and degradation, it is sufficient to block or occlude at least one of the S9 regulatory site and the R26 regulatory site of AP3D1.

Some embodiments of the present disclosure, based on the regulatory mechanism of the negative regulator TGF-β on STING transport and degradation, provide the application of AP3D1 as a drug target for in vitro screening of blockers. This approach identified two critical regulatory sites: the S9 regulatory site and the R26 regulatory site. Blocking peptides corresponding to the S9 regulatory site and the R26 regulatory site are synthesized to specifically prevent AP3D1-mediated inactivation of STING signaling by tumor microenvironment factors, thereby enabling blocking of at least one of the S9 regulatory site and the R26 regulatory site of AP3D1 to effectively restrict STING transport and degradation. Furthermore, the blocking peptides can synergize with various therapies—including chemotherapy, radiotherapy, a PARP inhibitor, and a STING agonist—to significantly activate intratumoral cGAS-STING signaling during treatment, thereby achieving more potent antitumor efficacy.

The present disclosure provides an application of the aforementioned blocking peptide in the preparation of a polypeptide formulation.

One or more embodiments of the present disclosure provide a polypeptide formulation, which may comprise the aforementioned blocking peptide.

The polypeptide formulation refers to a pharmaceutical preparation or a biological preparation in which a polypeptide serves as a primary active ingredient. In some embodiments, the polypeptide formulation is an injectable preparation.

In some embodiments, the polypeptide formulation may further comprise a pharmaceutically acceptable excipient.

The pharmaceutically acceptable excipient refers to an additional component used in the pharmaceutical preparation or the biological preparation, other than the aforementioned blocking peptide. The excipient plays an auxiliary role in the pharmaceutical preparation, ensuring stability, efficacy, safety, and applicability of the pharmaceutical preparation. For example, the excipient may include a filler, a binder, a stabilizer, a solubilizer, etc.

In some embodiments of the present disclosure, the blocking peptide in the polypeptide formulation may synergize with various treatment modalities such as chemotherapy, radiotherapy, a PARP inhibitor, a STING agonist, etc., thereby significantly activating intratumoral cGAS-STING signaling during treatment to achieve more effective antitumor efficacy.

One or more embodiments of the present disclosure provide a tumor-inhibiting composition. The tumor-inhibiting composition may include the aforementioned polypeptide formulation, and at least one of a PARP inhibitor and a STING agonist.

The tumor-inhibiting composition refers to a class of pharmaceuticals or biological preparations capable of inhibiting tumor growth, proliferation, or metastasis.

In some embodiments, the tumor targeted by the tumor-inhibiting composition includes an ovarian tumor, a breast tumor, or a colon tumor.

The Poly(ADP-ribose) polymerase (PARP) inhibitor refers to a class of anticancer drugs. PARP plays a crucial role in DNA repair, and the PARP inhibitor interferes with a DNA repair capacity of a tumor cell by inhibiting PARP function, thereby inducing tumor cell death.

In some embodiments, the PARP inhibitor may include niraparib or olaparib.

In some embodiments, the PARP inhibitor may also be other formulations. For example, the PARP inhibitor may further include rucaparib, talazoparib, fluzoparib, etc.

The stimulator of interferon genes (STING) agonist refers to a class of compounds or molecules capable of activating a STING signaling pathway. STING is a key protein in an innate immune system, and activation of the STING signaling pathway may induce the production of type I interferons (IFN-α/β) and other pro-inflammatory cytokines, thereby enhancing antitumor immune responses or antiviral immune responses.

In some embodiments, the STING agonist may include MSA-2.

In some embodiments, the STING agonist may be other formulations. For example, the STING agonist may include cyclic dinucleotides, MK-1454, DMXAA, etc.

In some embodiments of the present disclosure, the blocking peptide in the polypeptide formulation may effectively synergize with the PARP inhibitor and the STING agonist, thereby enabling the composition to exhibit excellent tumor-inhibiting effects. The tumor-inhibiting composition demonstrates superior in vivo tumor suppression efficacy, providing an effective approach for tumor inhibition and treatment.

One embodiment of the present disclosure provides an application of AP3D1 as a drug target for in vitro screening of blockers, where the drug target includes the S9 regulatory site and R26 regulatory site.

One embodiment of the present disclosure provides a blocking peptide capable of blocking at least one of the S9 regulatory site and the R26 regulatory site of AP3D1.

In some embodiments, the blocking peptide may include at least one of the following amino acid sequences or combinations thereof: A. The amino acid sequence set forth in at least one of SEQ ID NO: 1 and SEQ ID NO: 2; B. An amino acid sequence having 90%-99% sequence identity with at least one of SEQ ID NO: 1 and SEQ ID NO: 2; C. The amino acid sequence of A or B conjugated with a tag or a modifying group at the N-terminus, the C-terminus, or both the N-terminus and the C-terminus; D. An amino acid sequence derived from A, B or C modified with at least one of substitution, deletion, and addition of one or more amino acids.

One embodiment of the present disclosure provides an application of the blocking peptide in preparing a polypeptide formulation.

One embodiment of the present disclosure provides a polypeptide formulation comprising the aforementioned blocking peptide.

In some embodiments, the polypeptide formulation further comprises a pharmaceutically acceptable excipient.

One embodiment of the present disclosure provides a tumor-inhibiting composition comprising the aforementioned polypeptide formulation and further including at least one of a PARP inhibitor and a STING agonist.

In some embodiments, the PARP inhibitor is niraparib or olaparib.

In some embodiments, the STING agonist is MSA-2.

In some embodiments, the tumor is an ovarian tumor, a breast tumor, or a colon tumor.

The following examples provide more specific descriptions related to the aforementioned embodiments. Some content in these examples may also be replaced or combined with corresponding content from other examples to form new embodiments. Unless otherwise specified, experimental techniques in the following examples are conventional techniques. Unless otherwise specified, test materials used in the following examples were purchased from conventional biochemical reagent companies. All quantitative tests in the following examples were performed with three replicates, and results are presented as mean values. It should be understood that these examples are provided to better explain the present disclosure and are not intended to limit the present disclosure.

It should be noted that in the drawings, NC, Ctrl-pep, NC-pep, and NC-peptide all refer to the NC peptide; R26, R26 blocking peptide, R26 peptide, and R26-pep all refer to the R26-blocking peptide; S9, S9 blocking peptide, S9 peptide, and S9-pep all refer to the S9-blocking peptide.

### Example 1. In Vitro Effects of Blocking Peptides

This example designed and prepared blocking peptides capable of blocking the S9 regulatory site or the R26 regulatory sites of AP3D1, and investigated inhibitory effects of the blocking peptides on the STING-AP3D1 interaction and activating effects of the blocking peptides on the STING signaling pathway to validate the in vitro efficacy of the blocking peptides.

The blocking peptides used in this example were the S9-blocking peptide and the R26-blocking peptide, with amino acid sequences as shown in SEQ ID NO: 1 and SEQ ID NO: 2, respectively. A schematic structure of the blocking peptides is illustrated in FIG. 1. The peptides were synthesized by Anhui Guoping Pharmaceutical Co., Ltd. For further details on the S9-blocking peptide and the R26-blocking peptide, please refer to the preceding sections and corresponding descriptions.

The specific experimental procedures were as follows:

(1) Cell Culture: Human embryonic kidney 293 (HEK293) cells, purchased from the American Type Culture Collection (ATCC), were cultured in Dulbecco's Modified Eagle Medium (DMEM, Thermo Fisher Scientific) supplemented with 10% fetal bovine serum (FBS, Gibco) and penicillin-streptomycin solution (Pen-Strep, Thermo Fisher Scientific) at 37° C. in a 5% (v/v) CO₂ incubator to obtain experimental cells.

(2) Drug Treatment: Cells were transfected with a Myc-vector plasmid or a Myc-STING plasmid. The NC peptide (containing only the cell-penetrating peptide sequence Tyr Gly Arg Lys Lys Arg Arg Gln Arg Arg Arg Ala), the R26-blocking peptide, and the S9-blocking peptide were dissolved in phosphate-buffered saline (PBS) to prepare 10 mM stock solutions. Cells were treated with the peptide stocks for 24 hours, respectively, followed by stimulation with 10 ng/mL TGF-β for 3 hours to obtain cells for evaluating the STING-AP3D1 interaction.

For cells used to assess STING pathway activation, after TGF-β treatment, the cells were further stimulated with Poly(dA:dT) for 2 hours.

(3) Co-Immunoprecipitation (Co-IP) Assay: Cells from step (2) were lysed on ice for 15 minutes using a lysis buffer (20 mM Tris-HCl, pH 8.0, 100 mM NaCl, 1 mM EDTA, 0.5% Nonidet P-40) supplemented with protease inhibitors. The lysates were centrifuged at 15,000 rpm for 20 minutes to remove debris, and the supernatants were incubated with Anti-Myc magnetic beads overnight at 4° C. The resulting immunoprecipitated complexes were collected for further analysis.

(4) Immunoblotting (Western Blot) Assay: For immunoblotting, cell lysates or immunoprecipitates were denatured in a 1×SDS sample buffer at 95° C. for 10 minutes, followed by separation on 10% SDS-polyacrylamide gels. The resolved proteins were transferred to a polyvinylidene difluoride (PVDF) membrane, blocked with 5% non-fat milk for over 1 hour, and probed with a primary antibody at a recommended dilution. Detection was performed using an enhanced chemiluminescence (ECL) reagent (Thermo Fisher Scientific).

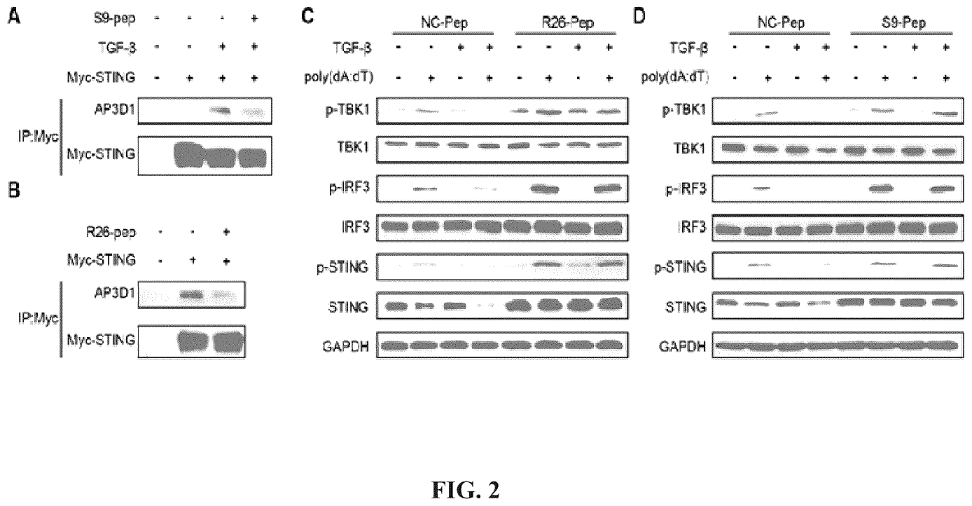

Immunoblot detection results of this example are shown in FIG. 2.

In FIG. 2, Panel A shows an interaction between STING and AP3D1 after blocking with the S9-blocking peptide; Panel B shows an interaction between STING and AP3D1 after blocking with the R26-blocking peptide; Panel C shows comparison of STING degradation levels between the NC peptide and the R26-blocking peptide; Panel D shows comparison of STING degradation levels between the NC peptide and the S9-blocking peptide.

As may be seen from Panels A and B of FIG. 2, the interaction between STING and AP3D1 was significantly attenuated after treatment with the S9-blocking peptide or the R26-blocking peptide, indicating that the S9-blocking peptide and the R26-blocking peptide effectively disrupt the STING-AP3D1 interaction in cells.

As may be seen from Panels C and D of FIG. 2, blocking with the S9-blocking peptide or the R26-blocking peptide reduced STING degradation and enhanced STING pathway activation.

These experimental results demonstrate that both the R26-blocking peptide and the S9-blocking peptide exhibit potent in vitro efficacy.

### Example 2. Effects of Polypeptide Drugs on Ovarian Cancer Development

This example evaluated the tumor growth in mice treated with an ovarian cancer-targeted drug Niraparib alone, the S9-blocking peptide alone, the R26-blocking peptide alone, or Niraparib combined with the S9-blocking peptide or the R26-blocking peptide, to assess the in vivo efficacy of the polypeptide drugs. The experimental procedure was as follows:

(1) Preparation of Polypeptide Drugs: The NC peptide, the S9-blocking peptide, and the R26-blocking peptide were dissolved in a PBS buffer to prepare 12.5 mg/mL intraperitoneal (IP) injection solutions, thus obtaining a protein-based R26 polypeptide drug and a protein-based S9 polypeptide drug.

(2) Ovarian Cancer Model Establishment: A total of 3×10⁶ luciferase-expressing Trp53^{-/-}Brca1^{-/-} ID8 cells were injected into the peritoneal cavity of 6- to 8-week-old female C57BL/6 mice to establish an advanced high-grade serous ovarian cancer model. The mice were randomly divided into 6 groups (Group A-Group F).

(3) Drug Administration (Starting 7 Days Post-Modeling)

Mice in Group A: The mice received daily intraperitoneal (IP) injections of the NC peptide at 12.5 mg/mL (100 μL per injection)

Mice in Group B: The mice received daily IP injections of the protein-based R26 polypeptide drug at 12.5 mg/mL (100 μL per injection).

Mice in Group C: The mice received daily IP injections of the protein-based S9 polypeptide drug at 12.5 mg/mL (100 μL per injection).

Mice in Group D: The mice received daily oral gavage of Niraparib at 10 mg/mL (100 μL per dose) co-administered with the NC peptide at 12.5 mg/mL (100 μL per injection).

Group E: The mice received daily oral gavage of Niraparib at 10 mg/mL (100 μL per dose) plus IP injection of the protein-based R26 polypeptide drug at 12.5 mg/mL (100 μL per injection).

Group F: The mice received daily oral gavage of Niraparib at 10 mg/mL (100 μL per dose) plus intraperitoneal injection of the protein-based S9 polypeptide drug at 12.5 mg/mL (100 μL per injection).

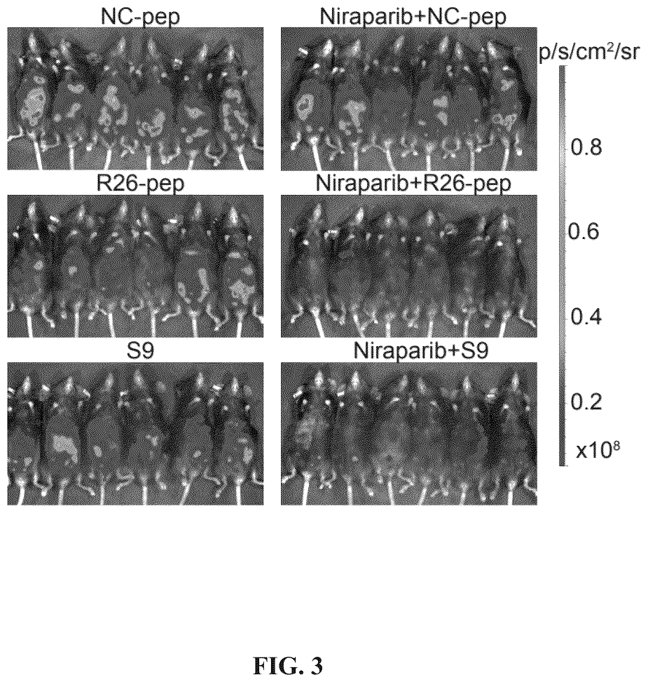

For each group of mice, a 2-day drug withdrawal period was implemented every 5 days of treatment. Tumor burden was monitored via small-animal imaging on the day of administration and at 1-3 weeks post-treatment. At 4 weeks post-treatment, tumor growth was assessed by in vivo small-animal imaging, where photon counts per second (p/s) in the abdominal region quantitatively reflected tumor cell counts. The mice were then euthanized, an ascites volume of each of the mice was collected, and a statistical analysis was performed using Student's t-test. Results are shown in FIGS. 3-5.

As may be seen from FIG. 3, the combination of Niraparib with the S9-blocking peptide (Niraparib+S9-pep) or the combination of Niraparib with the R26-blocking peptide (Niraparib+R26-pep) significantly inhibited ovarian tumor progression in the mice, compared to the treatment with Niraparib alone, the S9 blocking peptide alone, or the R26-blocking peptide alone.

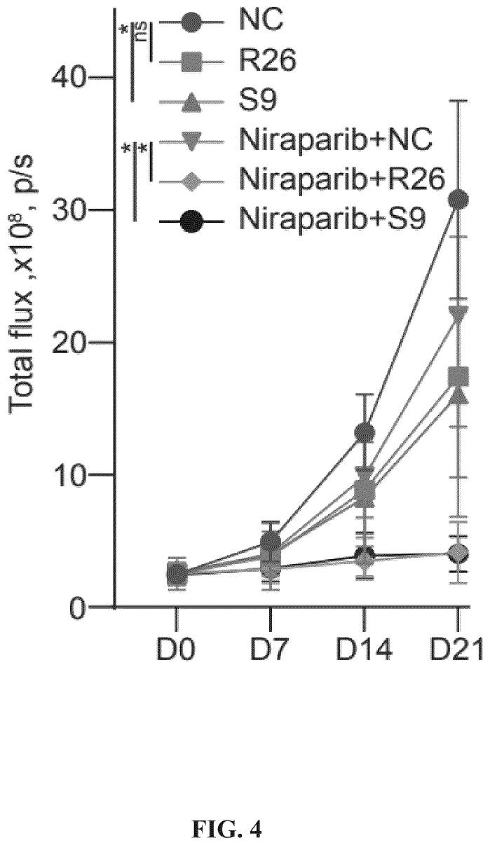

FIG. 4 shows that the tumor burden in mice treated with the combination of Niraparib+S9-pep or Niraparib+R26-pep was markedly reduced relative to the tumor burden in mice receiving Niraparib alone, the S9-blocking peptide alone, or the R26-blocking peptide alone.

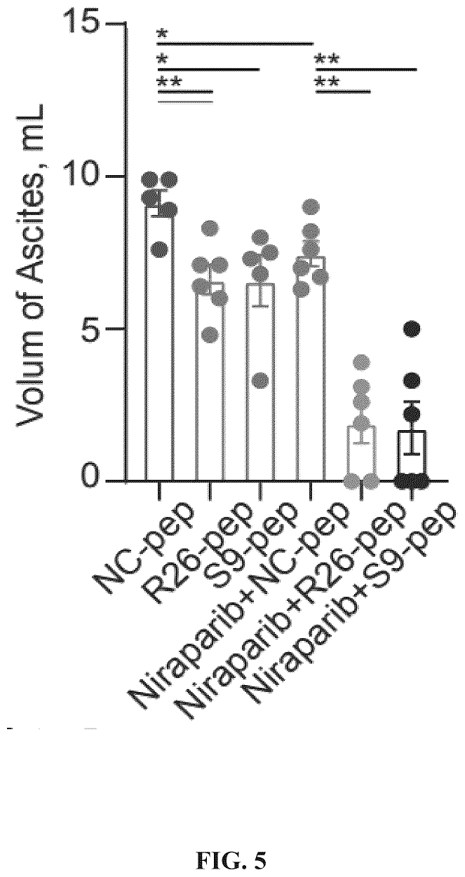

FIG. 5 reveals that the ascites volume in mice treated with the combination of Niraparib+S9-pep or Niraparib+R26-pep was significantly lower than in mice receiving Niraparib alone, the S9-blocking peptide alone, or the R26-blocking peptide alone.

### Example 3. Effects of Polypeptide Drugs on Breast Cancer Development

This example evaluated tumor growth in mice treated with a breast cancer-targeted drug Olaparib alone, the S9-blocking peptide alone, the R26-blocking peptide alone, or Olaparib combined with the S9-blocking peptide or the R26-blocking peptide, to assess the in vivo efficacy of the polypeptide drugs. The experimental procedure was as follows:

(1) Preparation of Polypeptide Drugs: The NC peptide, the S9-blocking peptide, and the R26-blocking peptide were dissolved in a PBS buffer to prepare 12.5 mg/mL intraperitoneal (IP) injection solutions, thus obtaining a protein-based R26 polypeptide drug and a protein-based S9 polypeptide drug.

(2) Orthotopic Breast Cancer Model Establishment: A total of 2×10⁵ Brca1^{-/-} EO771 cells were injected into the mammary fat pads of 6- to 8-week-old female C57BL/6 mice to establish an orthotopic breast cancer model. The mice were randomly divided into 6 groups (Group A-Group F).

(3) Drug Administration (Starting 3 Days Post-Modeling)

Mice in Group A: The mice received daily intraperitoneal (IP) injections of the NC peptide at 12.5 mg/mL (100 μL per injection)

Mice in Group B: The mice received daily IP injections of the protein-based R26 polypeptide drug at 12.5 mg/mL (100 μL per injection).

Mice in Group C: The mice received daily IP injections of the protein-based S9 polypeptide drug at 12.5 mg/mL (100 μL per injection).

Mice in Group D: The mice received daily oral gavage of Olaparib at 10 mg/mL (100 μL per dose) co-administered with the NC peptide at 12.5 mg/mL (100 μL per injection).

Mice in Group E: The mice received daily oral gavage of Olaparib at 10 mg/mL (100 μL per dose) plus IP injection of the protein-based R26 polypeptide drug at 12.5 mg/mL (100 μL per injection).

Mice in Group F: The mice received daily oral gavage of Olaparib at 10 mg/mL (100 μL per dose) plus intraperitoneal injection of the protein-based S9 polypeptide drug at 12.5 mg/mL (100 μL per injection).

For each group of mice, a 2-day drug withdrawal period was implemented every 5 days of treatment. Tumor volume was assessed every other day using the formula: L×W²×½ (wherein L denotes a length of the tumor, and W denotes a width of the tumor). On day 26 post-treatment, the mice were euthanized, and the tumor growth and a tumor weight of each of the mice were observed and measured for evaluation.

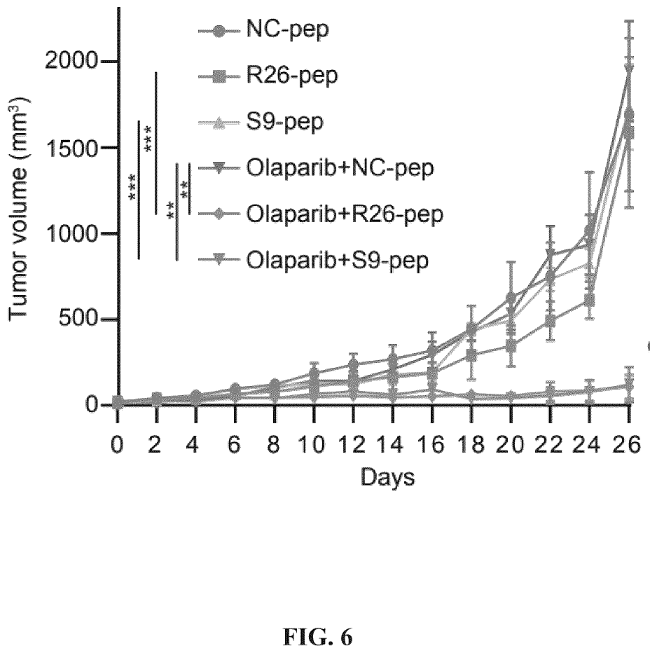

Experimental results of this example are shown in FIGS. 6-8.

FIG. 6 demonstrates that compared to Olaparib monotherapy, the S9-blocking peptide alone, or the R26-blocking peptide alone, the groups treated with the combination of Olaparib with the S9-blocking peptide (Olaparib+S9-pep) and the combination of Olaparib with the R26-blocking peptide (Olaparib+R26-pep) significantly inhibited breast tumor progression in mice.

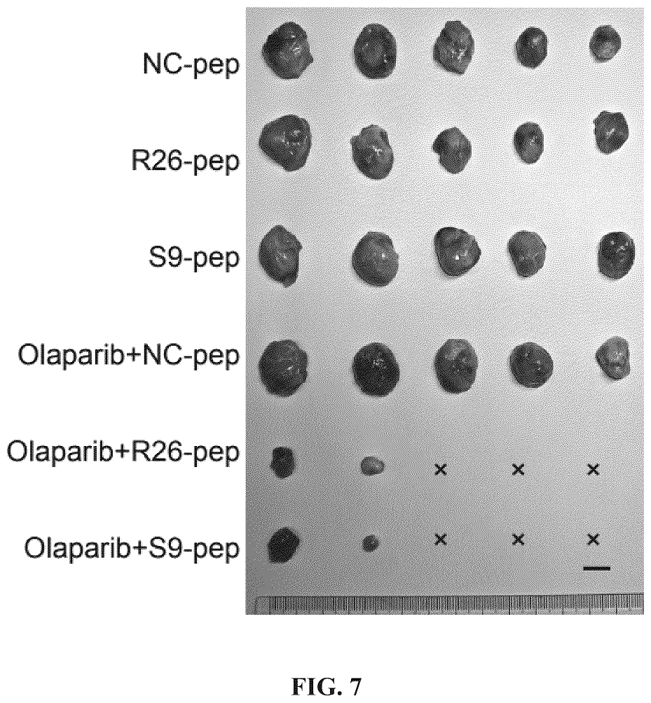

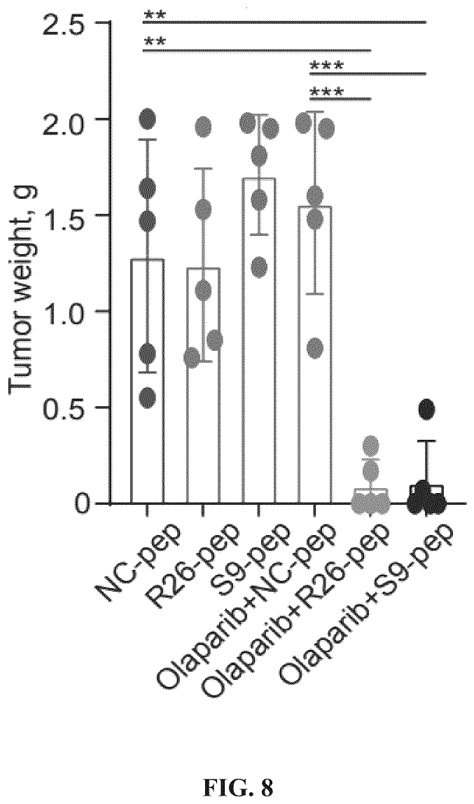

FIGS. 7 and 8 show that a tumor size and a tumor weight in the groups treated with the combination of Olaparib+S9-pep and the combination of Olaparib+R26-pep were significantly smaller/lower than a tumor size and a tumor weight in the monotherapy groups (the groups treated with Olaparib alone, the S9-blocking peptide alone, or the R26-blocking peptide alone).

### Example 4. Effects of Polypeptide Drugs on Subcutaneous Colon Cancer Development

This example evaluated tumor growth in mice treated with a STING agonist MSA-2 alone, the S9-blocking peptide alone, the R26-blocking peptide alone, or the STING agonist MSA-2 combined with the S9-blocking peptide or the R26-blocking peptide, to assess the in vivo efficacy of the polypeptide drugs. The experimental procedure was as follows:

(1) Preparation of Polypeptide Drugs: The NC peptide, the S9-blocking peptide, and the R26-blocking peptide were dissolved in a PBS buffer to prepare 12.5 mg/mL intraperitoneal (IP) injection solutions, thus obtaining a protein-based R26 polypeptide drug and a protein-based S9 polypeptide drug.

(2) Subcutaneous Colon Cancer Model Establishment: A total of 2×10⁵ CT26 cells were injected into the flanks of 6- to 8-week-old female BALB/c mice to establish a subcutaneous colon cancer model. The mice were randomly divided into 6 groups (Group A-Group F).

(3) Drug Administration (Starting 3 Days Post-Modeling):

Mice in Group A: The mice received daily intraperitoneal (IP) injections of the NC peptide at 12.5 mg/mL (100 μL per injection)

Mice in Group B: The mice received daily IP injections of the protein-based R26 polypeptide drug at 12.5 mg/mL (100 μL per injection).

Mice in Group C: The mice received daily IP injections of the protein-based S9 polypeptide drug at 12.5 mg/mL (100 μL per injection).

Mice in Group D: The mice received oral gavage of the STING agonist MSA-2 at 12 mg/mL (100 μL) co-administered with the NC peptide at 12.5 mg/mL (100 μL per injection) every five days.

Mice in Group E: The mice received daily oral gavage of the STING agonist MSA-2 at 12 mg/mL (100 μL per dose) plus IP injection of the protein-based R26 polypeptide drug at 12.5 mg/mL (100 μL per injection) every five days.

Mice in Group F: The mice received daily oral gavage of the STING agonist MSA-2 at 12 mg/mL (100 μL per dose) plus intraperitoneal injection of the protein-based S9 polypeptide drug at 12.5 mg/mL (100 μL per injection) every five days.

For each group of mice, a 2-day drug withdrawal period was implemented every 5 days of treatment. Tumor volume was assessed every other day using the formula: L×W²×½ (wherein L denotes a length of the tumor, and W denotes a width of the tumor). On day 26 post-treatment, the mice were euthanized, and the tumor growth and a tumor weight of each of the mice were observed and measured for evaluation.

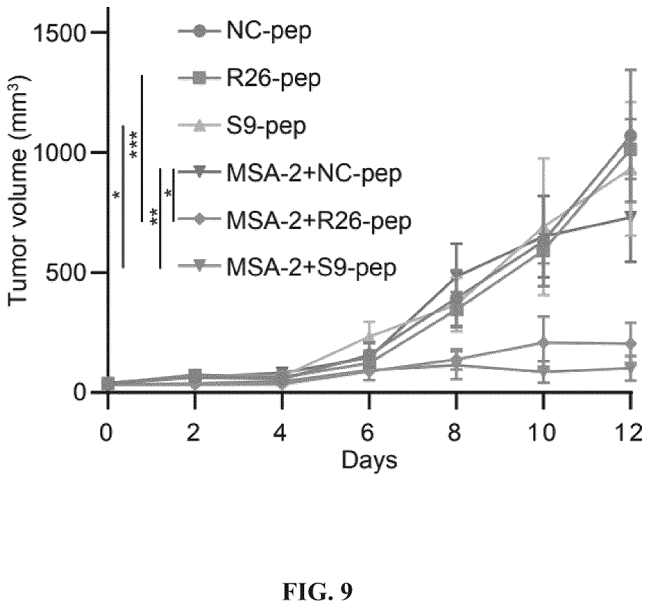

Experimental results of this example are shown in FIGS. 9-11.

FIG. 9 demonstrates that compared to administering the STING agonist MSA-2 alone, the S9-blocking peptide alone, or the R26-blocking peptide alone, the groups treated with the combination of the STING agonist MSA-2 with the S9-blocking peptide (MSA-2+S9-pep) and the combination of the STING agonist MSA-2 with the S26-blocking peptide (MSA-2+R26-pep) significantly inhibited subcutaneous colon tumor progression in mice.

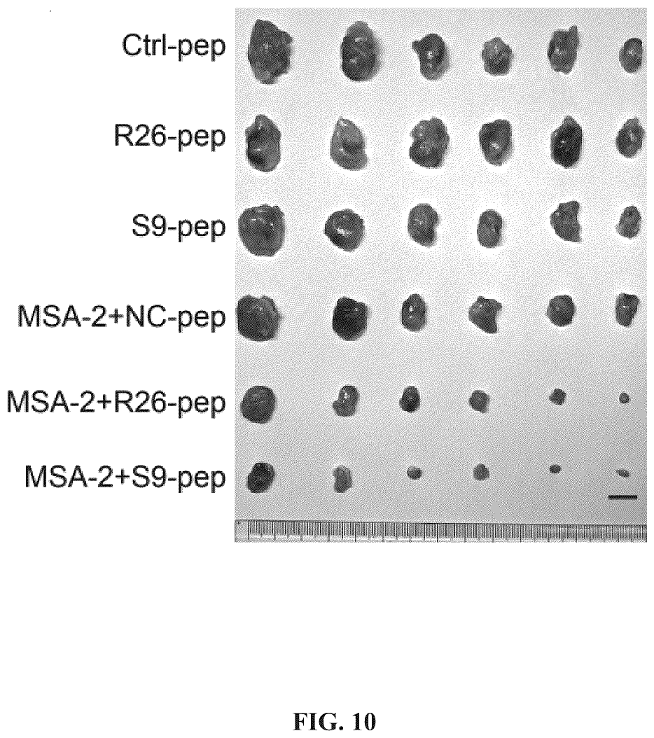

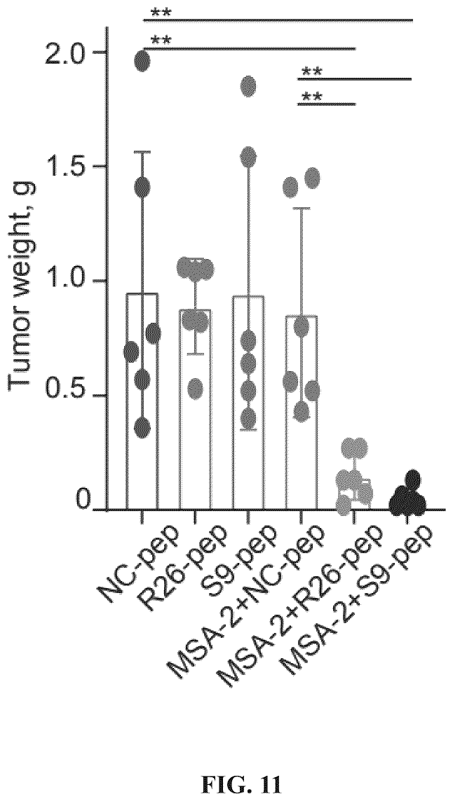

FIGS. 10 and 11 show that a tumor size and a tumor weight in the groups treated with the combination of MSA-2+S9-pep and the combination of MSA-2+R26-pep were significantly smaller/lower than a tumor size and a tumor weight in the monotherapy groups (the groups treated with the STING agonist MSA-2 alone, the S9-blocking peptide alone, or the R26-blocking peptide alone).

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

As indicated in the present disclosure and in the claims, the singular forms “a,” “an,” and “the” may be intended to include the plural forms as well, unless the context clearly indicates otherwise. In general, the terms “comprise,” “comprises,” and/or “comprising,” “include,” “includes,” and/or “including,” when used in this disclosure, specify the presence of stated features, integers, steps, operations, elements, and/or components, but do not preclude the presence or addition of one or more other features, integers, steps, operations, elements, components, and/or groups thereof. In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

What is claimed is:

1. A blocking peptide, capable of blocking at least one of an S9 regulatory site and an R26 regulatory site of Adaptor-Related Protein Complex 3 Subunit Delta 1 (AP3D1).

2. The blocking peptide of claim 1, comprising:
(a) an amino acid sequence having 90% to 99% sequence identity to at least one of:
(1) the amino acid sequence set forth in SEQ ID NO: 1, and
(2) the amino acid sequence set forth in SEQ ID NO: 2;
or
(b) a combination of amino acid sequences, wherein each sequence in the combination independently satisfies (a).

3. The blocking peptide of claim 2, comprising:
(a) an amino acid sequence selected from at least one of:
(1) the amino acid sequence set forth in SEQ ID NO: 1, and
(2) the amino acid sequence set forth in SEQ ID NO: 2;
or
(b) a combination of amino acid sequences, wherein each sequence in the combination independently satisfies (a).

4. The blocking peptide of claim 3, comprising:
(a) an amino acid sequence selected from at least one of:
(1) the amino acid sequence set forth in SEQ ID NO: 1, and
(2) the amino acid sequence set forth in SEQ ID NO: 2,
wherein the selected amino acid sequence has one or more modifications selected from:
(i) substitution of one or more amino acids,
(ii) deletion of one or more amino acids, and
(iii) addition of one or more amino acids;
or
(b) a combination of amino acid sequences, wherein each sequence in the combination independently satisfies (a).

5. The blocking peptide of claim 3, comprising:
(a) an amino acid sequence selected from at least one of:
(1) the amino acid sequence set forth in SEQ ID NO: 1,
(2) the amino acid sequence set forth in SEQ ID NO: 2, and
(3) the amino acid sequence set forth in SEQ ID NO: 3, which is included within
at least one of SEQ ID NO: 1 and SEQ ID NO: 2,
wherein the selected amino acid sequence is conjugated with:
a tag or a modifying group at an N-terminus, a C-terminus, or both the N-terminus and the C-terminus;
or
(b) a combination of amino acid sequences each independently selected from (a)(1)-(3) and conjugated as defined in (a).

6. The blocking peptide of claim 3, comprising:
(a) an amino acid sequence selected from at least one of:
(1) the amino acid sequence set forth in SEQ ID NO: 1, and
(2) the amino acid sequence set forth in SEQ ID NO: 2,
wherein the selected amino acid sequence has one or more modifications selected from:
(i) substitution of one or more amino acids,
(ii) deletion of one or more amino acids, and
(iii) addition of one or more amino acids, and
the modified sequence is conjugated with:
a tag or a modifying group at an N-terminus, a C-terminus, or both the N-terminus and the C-terminus;
or
(b) a combination of amino acid sequences each independently satisfying the conditions of (a).

7. A peptide formulation, comprising the blocking peptide of claim 2.

8. The peptide formulation of claim 7, further comprising a pharmaceutically acceptable excipient.

9. A tumor-inhibiting composition, comprising:
(a) the polypeptide formulation of claim 7; and
(b) a Poly(ADP-ribose) polymerase (PARP) inhibitor or a stimulator of interferon genes (STING) agonist.

10. The tumor-inhibiting composition of claim 9, wherein the PARP inhibitor includes niraparib or olaparib.

11. The tumor-inhibiting composition of claim 9, wherein the STING agonist includes MSA-2.

12. The tumor-inhibiting composition of claim 9, wherein the tumor includes an ovarian tumor, a breast tumor, or a colon tumor.


SEQUENCE LISTING (3 sequences)

SEQ ID NO: 1 - protein, 23 aa, synthetic construct
YGRKKRRQRRRAKMVKGSIDRMF

SEQ ID NO: 2 - protein, 23 aa, synthetic construct
YGRKKRRQRRRALVRGIRNHKED

SEQ ID NO: 3 - protein, 12 aa, synthetic construct
YGRKKRRQRRRA